(19) United States
(12) Patent Application Publication
(10) Pub. No.: US 2025/0152492 A1
(43) Pub. Date: May 15, 2025

(54) Deodorant Compositions

(71) Applicant: Conopco, Inc., d/b/a UNILEVER, Englewood Cliffs, NJ (US)

(72) Inventors: Tammy Baskerville, Leeds (GB); Natasha Suzanne Rigby Day, Leeds (GB); Joy Rachel McWalter, Leeds (GB); Alan Philip Rowland, Leeds (GB)

(21) Appl. No.: 18/942,165

(22) Filed: Nov. 8, 2024

(30) Foreign Application Priority Data
Nov. 13, 2023 (EP) 23209553.9

(51) Int. Cl. A61K 8/58; A61K 8/06; A61K 8/365; A61Q 15/00

(52) U.S. Cl. CPC A61K 8/58; A61K 8/062; A61K 8/365; A61Q 15/00

(57) ABSTRACT

A highly effective skin care deodorant composition comprising zinc neodecanoate; lactic acid or a salt thereof; and a cosmetically acceptable carrier.

### FIELD OF THE INVENTION

The invention is concerned with deodorant compositions comprising a deodorant active and a skin care active.

### BACKGROUND OF THE INVENTION

Deodorant compositions comprising zinc-based actives are known in the art. Examples of publications disclosing such compositions are given below.

Zinc ricinoleate is a particularly well known zinc-based deodorant. EP 3878431 A1 (Hyteck, 2021) discloses stable emulsion deodorant compositions comprising zinc ricinoleate.

WO 18/087147 A1 (Givaudan, 2018) discloses deodorant compositions comprising zinc neodecanoate and fragrance.

WO 18/087148 A1 (Givaudan, 2018) discloses deodorant compositions comprising zinc neodecanoate and aluminium chlorohydrate.

### SUMMARY OF THE INVENTION

The invention is concerned with deodorant compositions comprising a specific deodorant active and lactic acid, which may serve as a skin care active.

It is an object of the invention to provide a deodorant composition that delivers both good deodorancy benefits and also good skin care benefits.

It is a further object of the invention to provide a deodorant composition having good rheological stability.

It is a further object of the invention to provide a deodorant composition having good compatibility between components; in particular, good compatibility between a deodorant active and a skin care active such that neither unduly affects the performance of the other.

In a first aspect of the invention, there is provided a deodorant composition comprising (i) zinc neodecanoate at from 0.1 to 20%, (ii) lactic acid and (iii) a cosmetically acceptable carrier, wherein the ratio of the zinc neodecanoate to the lactic acid is from 1:4 to 10:1.

In a second aspect of the invention, there is provided a method of manufacture of a deodorant composition according to the first aspect of the invention.

In a third aspect of the invention, there is provided a cosmetic method of obtaining a deodorancy benefit comprising the topical application to the surface of the human body of a deodorant composition according to the first aspect of the invention.

In a fourth aspect of the invention, there is provided the use of a deodorant composition according to the first aspect of the invention to deliver a deodorancy benefit and, preferably, a skin care benefit to the surface of the human body.

In a fifth aspect fourth aspect of the invention, closely related to the fourth aspect, there is provided the use of a deodorant composition according to the first aspect of the invention to give reduced malodourous volatile fatty acids and, preferably, a skin care benefit to the surface of the human body.

In a sixth aspect of the invention, closely related to the fourth aspect, there is provided the use of a deodorant composition according to the first aspect of the invention to give reduced malodourous thioalcohols and, preferably, a skin care benefit to the surface of the human body.

### DETAILED DESCRIPTION OF THE INVENTION

Herein, features expressed as “preferred” with regard to a particular aspect of the invention should be understood to be preferred with regard to each aspect of the invention (likewise, features expressed as “more preferred”, “particularly preferred” or “most preferred”). “Preferred” features aid the delivery of one or more of the objects of the invention.

Herein, preferred features of the invention are particularly preferred when used in combination with other preferred features (likewise, features expressed as “more preferred”, “particularly preferred” or “most preferred”).

Herein, “ambient conditions” refer to 20° C. and 1 atmosphere pressure, unless otherwise indicated.

Herein, “water insoluble” refers to materials having a solubility in water of less than 0.1 g/100 g, under ambient conditions.

Herein, all percentages, ratios and amounts are by weight, unless otherwise indicated.

Herein, references to “lactic acid” should be understood to mean “lactic acid or a salt thereof” when this is not already explicitly stated.

Herein, amounts and concentrations of ingredients are percentages by weight of the total composition, unless otherwise indicated.

Herein, references to amounts of components such as “deodorant active” or “rheological modifier” relate to the total amount of such components present in the composition.

Herein, the word “comprising” is intended to mean “including” but not necessarily “consisting of”, i.e., it is non-exhaustive.

### Herein, “Cosmetic” Methods and Compositions Should be Understood to Mean Non-Therapeutic Methods and Compositions, Respectively

Zinc neodecanoate is the deodorant active used in the present invention. Zinc neodecanoate is the zinc dicarboxylate salt of neodecanoic acid having the formula: [C₉H₂₀—CO·O]₂Zn.

Whilst other deodorant actives may be present, it is preferred that zinc neodecanoate is the predominant one, i.e. comprising than 50% by weight of all of the deodorant actives present.

Zinc neodecanoate is a deodorant active known to function by absorption of malodours emanating from the skin of the humans [see WO 18/087147 A1 (Givaudan, 2018) referred to above]. Important classes of such malodours are thioalcohols and volatile fatty acids (VFAs). The present invention stems at least in part from the observation that the absorption of these malodours by zinc neodecanoate is not unduly hindered by the presence of the lactic acid.

Hence, the present invention delivers the skin care benefits deriving from the lactic acid without undue reduction in the malodour absorption by the zinc neodecanoate.

The VFAs absorbed by zinc neodecanoate are largely short chain carboxylic acids, typically having up to about 7 carbon atoms. Isovaleric acid [(CH₃)₂CHCH₂CO₂H] is a typical example. Such VFAs are extremely odiferous and also include short chain carboxylic acids having hydroxyl groups, such as 3-hydroxy-3-methylhexanoic acid, sometimes abbreviated to 3H3MH or HMHA and having the structural formula CH₃CH₂CH₂C(CH₃)(OH)CH₂CO₂H.

The present invention is underpinned by one or two surprising observations:
first, the lactic acid is not absorbed onto the zinc neodecanoate to such an extent that it hinders absorption of thioalcohols onto the zinc neodecanoate nor the absorption of VFAs to a greater than acceptable extent;
secondly, the lactic acid is not absorbed by the zinc neodecanoate to an extent such that it cannot perform its function as a skin care active.

These observations are particularly surprising when one recognises the great molecular similarities between lactic acid and the VFAs found on the surface of the human body (which are known to be absorbed by zinc neodecanoate). Both are relatively short chain carboxylic acids, and both have a hydroxy group (lactic acid) or may have a hydroxy group (VFAs).

The zinc neodecanoate is present at from 0.1 to 20%, preferably at from 0.5 to 10% and more preferably at from 1 to 7% of the total composition.

The ratio of the zinc neodecanoate to the lactic acid is preferably from 1:4 to 10:1, more preferably from 1:2 to 6:1 and most preferably from 1:1 to 3:1. At these ratios, the performance of both components may be improved and further improved.

The lactic acid may serve as a skin care active, delivering a number of potential benefits on the skin, including gentle exfoliation, improved skin texture, and reducing hyperpigmentation. The present invention also involves the possibility of a positive influence on the deodorancy performance of zinc neodecanoate by the lactic acid.

The level of lactic acid or salt thereof used in compositions of the invention is preferably from 0.1 to 20%, more preferably at from 0.5 to 10% and most preferably at from 1 to 7% of the total composition.

The cosmetically acceptable carrier may be selected from any of those known in the art. In most preferred embodiments, the cosmetically acceptable carrier comprises a carrier oil, i.e. a water insoluble substance which is liquid under ambient conditions.

In some embodiments, water can also be a suitable carrier material.

In some preferred embodiments the deodorant composition is in the form of an emulsion, in particular an oil-in-water emulsion. Emulsions compositions are particularly preferred because the zinc neodecanoate sits in the oil phase and the lactic acid sits in the aqueous phase, minimising interactions between the two.

When an oil-in-water emulsion composition is employed, the aqueous is typically the predominant phase, preferably comprising greater than 60%, more preferably greater than 75%, and most preferably greater than 80% by weight of the total composition.

A preferred additional component in compositions of the invention, and particularly in emulsion compositions, is a humectant selected from glycerol and/or a PEG oligomer of weight average molecular weight of from 180 to 420. Glycerol is an especially preferred humectant. The humectant may enhance the skin care properties of the composition.

When employed, the humectant is present in the total composition at a preferred level of from 1 to 20%, more preferably from 2 to 12% and most preferably from 3 to 10%, these levels being particularly relevant when the humectant is glycerol.

A preferred additional component in compositions of the invention, and particularly in emulsion compositions, is a water soluble vitamin, such as Vitamin B3 or niacinamide. Such vitamins are present at a level at a preferred level of from 0.1 to 8%, more preferably from 0.5 to 6% and most preferably from 1 to 5% by weight of the total composition. Such vitamins may enhance the skin care properties of the composition.

A preferred additional component in compositions of the invention is a fragrance oil, alternatively known as a perfume oil. The fragrance oil may comprise a single fragrance component or, more commonly, a plurality of fragrance components. Herein, fragrance oils impart an odour, preferably a pleasant odour, to the composition. Preferably, the fragrance oil imparts a pleasant odour to the surface of the human body the composition is applied to the same.

The amount of fragrance oil in the composition is preferably up to 3% advantageously is at least 0.5% and particularly preferably from 0.8% to 2%.

In certain embodiments, it is preferred to include an oil, other than a fragrance oil, that has a relatively low viscosity, by which is meant less 250 cS (mm²·s⁻¹). Such oils, termed “non-fragrance oils”, can improve the sensory properties of the composition on application and can lead to other benefits such as emolliency.

Preferred non-fragrance oils are alkyl ether oils having a boiling point of above 100° C. and especially above 150° C., including polyalkyleneglycol alkyl ethers. Such ethers desirably comprise between 10 and 20 ethylene glycol or propylene glycol units and the alkyl group commonly contains from 4 to 20 carbon atoms. The preferred ether oils include polypropylene glycol alkyl ethers such as PPG-14-butylether and PPG-15-stearyl ether.

Other preferred non-fragrance are triglyceride oils. The triglyceride oils commonly comprise the alkyl residues of aliphatic C₇ to C₁₈ acids, the total number of carbon atoms being selected in conjunction with the extent of olefinic unsaturation and/or branching to enable the triglyceride to be liquid at 20° C. Particularly preferably, in the triglyceride oil the alkyl residues are linear C₁₈ groups having one, two or three olefinic degrees of unsaturation, two or three being optionally conjugated, many of which are extractable from plants (or their synthetic analogues), including triglycerides of oleic acid, linoleic acid, conjugated linoleic acids, linolenic acid, petroselenic acid, ricinoleic acid, linolenelaidic acid, trans 7-octadecenoic acid, parinaric acid, pinolenic acid, punicic acid, petroselenic acid and stearidonic acid.

Suitable oils can include those derived from unsaturated C₁₈ acids, including coriander seed oil, impatiens balsimina seed oil, parinarium laurinarium kernel fat oil, sabastiana brasilinensis seed oil, dehydrated castor seed oil, borage seed oil, evening primrose oil, aquilegia vulgaris oil, sunflower (seed) oil and safflower oil. Other suitable oils are obtainable from hemp, and maize corn oil. An especially preferred oil by virtue of its characteristics is sunflower (seed) oil.

Further suitable oils, that can also be emollient oils, comprise alkyl or alkyl-aryl ester oils having a boiling point of above 150° C. (and a melting point of below 20° C.). Such ester oils include oils containing one or two alkyl groups of 12 to 24 carbon atoms length, including isopropyl myristate, isopropyl palmitate and myristyl palmitate. Other non-volatile ester oils include alkyl or aryl benzoates such C₁₂₋₁₅ alkyl benzoate, for example Finsolv TN™ or Finsolv Sun™.

Emulsion compositions according to the invention are preferably stabilised with a nonionic emulsifier system. Preferably, the nonionic emulsifier system comprises a low HLB ethoxylated alcohol (HLB less than 8) and a high HLB ethoxylated alcohol (HLB more than 12). More preferably, the low HLB ethoxylated alcohol has a HLB less than 5 and the high HLB ethoxylated alcohol has a HLB of more than 15.

When a nonionic emulsifier system as described in the paragraph immediately above is employed, it is preferred that the ratio of the low HLB ethoxylated alcohol to the high HLB ethoxylated alcohol is from 1:1 to 1:10 and more preferably from 2:3 to 1:8.

An especially preferred emulsifier nonionic system comprises Steareth-2 and Steareth-20. Steareth-2 has a HLB of 4.9 and Steareth-20 has a HLB of 15.3.

The non-ionic emulsifier system preferably comprises from 0.5 to 10%, more preferably from 1 to 8%, and most preferably from 1 to 6%, all relative to the total composition.

Emulsion compositions according to the invention are preferably thickened with a rheology modifier.

Preferred rheology modifiers are C16-C22 linear fatty alcohols and/or C16-C22 fatty acid glycerides. It is particular preferred to use both a C16-C22 linear fatty alcohol and a C16-C22 fatty acid glyceride.

When a C16-C22 linear fatty alcohol is employed, it is preferably a C18 linear fatty alcohol and more preferable stearyl alcohol.

When employed, C16-C22 linear fatty alcohol is preferably used at from 1 to 12% of the total composition and more preferably at from 2 to 7% by weight of the total composition.

When employed, C16-C22 fatty acid glyceride is preferably rich in monoglyceride content, meaning that it comprises at least 25% C16-C22 fatty acid monoglyceride and more preferably at least 30% C16-C22 fatty acid monoglyceride.

When employed, C16-C22 glyceride preferably comprises at least 25% glyceryl monostearate and more preferably at least 30% of said monoglyceride.

When employed, C16-C22 fatty acid glyceride is preferably used at from 1 to 12% of the total composition and more preferably at from 2 to 7% by weight of the total composition.

When both a C16-C22 linear fatty alcohol and a C16-C22 fatty acid glyceride are employed, including combinations of the preferred and more preferred selections indicated above, there are certain preferred ratios between the two. The ratio of the C16-C22 linear fatty alcohol to the C16-C22 fatty acid glyceride is preferably from 1:4 to 4:1, more preferably from 1:3 to 3:1 and most preferably from 1:2 to 2:1, by weight.

When employed, the total content of the rheological modifier consisting of a C16-C22 linear fatty alcohol and/or a C16-C22 fatty acid glyceride is preferably from 4 to 20%, more preferably from 5 to 15% and most preferably from 6 to 13%, by weight of the total composition.

In some preferred embodiments of the invention, compositions of the invention are typically shear-thinning, i.e. they are non-Newtonian fluids that decrease in viscosity as the shear rate is increased. At a shear rate of /10s, they have a viscosity of from 10,000 cPs to 300,000 cPs at 10/s, measured at 20° C. In SI units, this equates to from 10,000 mPa·s to 3000,000 mP·s at 10/s and 20° C. Preferably, they have a viscosity of from 25,000 cPs to 300,000 cPs at 10/s and more preferably a viscosity of from 50,000 cPs to 250,000 cPs at 10/s, all measured at 20° C.

Herein, the abbreviation mP·s is short for the SI unit of viscosity millipascal·second.

Other components that may be included in compositions according to the invention including those described in the following paragraphs.

Wash-off agents may be included, often in an amount of up to 10%, to assist in the removal of the formulation from skin or clothing. Such wash-off agents are typically non-ionic surfactants such as esters or ethers containing a C₈ to C₂₂ alkyl moiety and a hydrophilic moiety comprising a polyoxyalkylene group (POE or POP).

Skin-feel improvers (e.g. talc or finely divided high molecular weight polyethylene), may be included, typically in an amount from 1 up to 10%.

A highly preferred optional component is a preservative system, such as sodium benzoate/citric acid and/or an antioxidant such BHT (butyl hydroxy toluene), each typically in an amount of from 0.01 to 0.5%.

The method of manufacture of the emulsion compositions that are according to the invention, including preferred variants thereof, involves the following steps.
A first vessel is filled with aqueous phase components, including water, the lactic acid and any other water soluble components, such as a water soluble emollient, vitamin(s) and/or preservative agent(s).
A second vessel is filled with the nonionic emulsifier system and the oil phase components including zinc neodecanoate and a rheology modifier.
Optionally but preferably, a third vessel is filled with further water and, optionally, fragrance encapsulates.
The first vessel and second vessel are heated to a temperature of 75° C. or greater and the components are allowed to dissolve and homogenise, preferably with stirring.
The contents of the first vessel and second vessel are then mixed under shear, the contents of the second vessel being preferably added to the stirred contents of the first vessel, preferably into a vortex created in the contents of the first vessel by the stirring thereof.
Optionally but preferably, the contents of the third vessel are then added at no more than ambient temperature in order to quench (reduce) the temperature of the mixture described above.
Preferably, the mixture described above is cooled to less than 50° C. before fragrance is added and stirred in to emulsify.
Finally, the mixture is subjected to high shear in order to build its viscosity. A shear rate of at least 1500 rpm is preferred, more preferably at least 2500 rpm and most preferably at least 3500 rpm, in order to build its viscosity to from 10,000 cPs to 300,000 cPs/s at 10/s and 20° C.

### EXAMPLES

The following Examples are illustrative of the invention and non-limiting as to its scope.

Examples according to the invention are indicated by numbers and Comparative Examples are indicated by letters.

The compositions labelled A and 1 in Table 1 were prepared by the following method. The term “batch” refers to the batch or sample of the mixture being prepared.

Into a main vessel, the following components were added: water, glycerol, niacinamide (Comparative Example A only) and sodium benzoate and lactic acid (Example 1 only). This mixture was heated to 85° C. and homogenised.

Into a first side-pot, the following components were added: the glyceryl stearate, stearyl alcohol, cetearyl alcohol (Example 1 only), steareth-2, steareth-20, PPG-15 stearyl ether (Comparative

Example A only), antioxidant, and zinc neodecanoate. This mixture was also heated to 85° C. and homogenised.

Into a second side-pot, further water and the phenoxyethanol and fragrance encapsulates (Comparative Example A only) were placed.

The stir speed in the main vessel was increased to form a vortex and the contents of first side-pot were added into the vortex and the mixture was allowed to mix for sufficient time to give good emulsification throughout the batch. The contents of the second side-pot (at ambient temperature) were then added to start cooling the batch. The batch was cooled to 43° C.

The fragrance was then added and stirring continued for a further 5 minutes.

Finally, the batch was transferred to a high shear mixture and sheared at 3700 rpm for 5 minutes.

Regarding the raw materials:
The glyceryl stearate comprised 40% glyceryl monostearate.
The antioxidant used was pentaerythrityl tetra-di-t-butyl hydrocinnamate.
The zinc neodecanoate also contained 20% isopropyl myristate.

TABLE 1

| | Examples | | | |
|---|---|---|---|---|
| | A | 1 | B | 2 |
| | % by wt. | | Wt. added (g) | |
| Glycerol | 3 | — | — | — |
| Glyceryl stearate | 3 | 7.5 | — | — |
| Stearyl alcohol | 5 | 3.5 | — | — |
| Cetearyl alcohol | — | 1 | — | — |
| Steareth-2 | 1 | 0.19 | — | — |
| Steareth-20 | 2 | 1.31 | — | — |
| PPG-15 stearyl ether | 3 | — | — | — |
| Antioxidant | 0.05 | — | — | — |
| Fragrance encapsulates | 0.75 | — | — | — |
| Zinc neodecanoate | 4 | 4 | 0.02 | 0.02 |
| Phenoxyethanol | 0.5 | — | — | — |
| Sodium benzoate | — | 0.5 | — | — |
| Fragrance | 1.3 | 1.3 | — | — |
| Niacinamide | 3 | — | — | — |
| Lactic acid | — | 2.2 | — | 0.02 |
| Water | To 100 | To 100 | — | — |
| Thioalcohol reduction (%) | 78 | 77 | 56 | 61 |
| VFA reduction (%) | 70 | 46 | 43 | 32 |

Malodour absorption by these compositions was assessed w.r.t. a thioalcohol malodour (3-mercapto-1-hexanol) and a volatile fatty acid (VFA) (isovaleric acid). The following procedure was used.

A malodour stock solution was prepared containing approximately 1000 ppm 3-mercapto-1-hexanol and approximately 5000 ppm isovaleric acid in water. Using GCMS, the peak areas corresponding to each of these malodorants was measured for duplicate 0.5 ml samples.

Test solutions for Comparative Example A and Example 1 were prepared by mixing 0.10 g of the Example composition with 0.5 ml of the malodour stock solution. Duplicate samples of the test solutions were analysed shortly after making* using GCMS, as above, to find the peak areas corresponding to the thioalcohol and VFA in the samples. *No more than a few minutes was allowed between the preparation of the Examples and their analysis—the odour absorption is quite rapid. With Comparative Example A, the thioalcohol malodour was reduced by 78% and the VFA malodour was reduced by 70%.

The reduction in thioalcohol or VFA malodour for each sample was calculated as:

100(1−[average peak area for malodorant in test solution divided by average peak area for malodorant in stock solution]).

Test solutions for Comparative Example B and Example 2 were much simpler. For Comparative Example B, the test solution consisted of 0.5 ml of the malodour stock solution plus 0.02 g of the zinc neodecanoate raw material as described above Table 1. For Example 2, the test solution consisted of 0.5 ml of the malodour stock solution plus 0.02 g of the zinc neodecanoate raw material as described above Table 1 plus 0.02 g of lactic acid. The reduction in thioalcohol or VFA malodour for these samples was calculated in a manner analogous to that described immediately above.

The results of the malodour absorption measurements are given at the bottom of Table 1.

Comparing Example 1 with Comparative Example A, it may be seen that the thioalcohol reduction is comparable and the VFA reduction is not overly impacted by the presence of the lactic acid. Comparing Example 2 with Comparative Example B, it may be seen that the thioalcohol reduction is marginally increased and the VFA reduction is again not overly impacted by the presence of the lactic acid. These are surprising observation for both malodorants.

1. A deodorant composition comprising (i) zinc neodecanoate at from 0.1 to 20% by weight; (ii) lactic acid or a salt thereof; and (iii) a cosmetically acceptable carrier, wherein the weight ratio of the zinc neodecanoate to the lactic acid is from 1:4 to 10:1.

2. The deodorant composition according to claim 1, wherein the deodorant composition is an oil-in-water emulsion.

3. The deodorant composition according to claim 1, wherein the deodorant composition comprises a rheology modifier and has a viscosity of at least 10,000 mPa·s to 300,000 mPa·s at 10/s and 20° C.

4. The deodorant composition according to claim 3, wherein the rheology modifier comprises a C16-C22 linear fatty alcohol.

5. The deodorant composition according to claim 3, wherein the rheology modifier comprises a C16-C22 fatty acid glyceride.

6. The deodorant composition according to claim 1, further comprising a nonionic emulsifier system.

7. The deodorant composition according to claim 6, wherein the nonionic emulsifier system comprises an ethoxylated alcohol of HLB less than 8 and an ethoxylated alcohol of HLB more than 12.

8. The deodorant composition according to claim 7, wherein the ratio of the ethoxylated alcohol having a HLB of less than 8 to the ethoxylated alcohol having a HLB of more than 12 is from 1:1 to 1:10 by weight.

9. The deodorant composition according to claim 1, comprising a humectant selected from glycerol and PEG oligomers having a molecular weight from 180 to 420

10. The deodorant composition according to claim 1, further comprising a fragrance.

11. A method of making an oil-in-water emulsion deodorant composition, comprising: (a) zinc neodecanoate at from 0.1 to 20% by weight: (b) lactic acid or a salt thereof; and (c) a cosmetically acceptable carrier, wherein the weight ratio of the zinc neodecanoate to the lactic acid is from 1:4 to 10:1, the method comprising
(i) filling a first vessel with aqueous phase components, including water, lactic acid or a salt thereof, and any other water soluble components;
(ii) filling a second vessel with a nonionic emulsifier system and oil phase components including zinc neodecanoate and a rheology modifier;
(iii) heating the first vessel and second vessel are heated to a temperature of 75° C. or greater, the components being allowed to dissolve and homogenise;
(iv) shear mixing a mixture of the contents of the first vessel and second vessel and
subjecting the mixture to high shear of at least 1500 rpm in order to build its viscosity to from 10,000 mPa·s to 300,000 mPa·s at 10/s and 20° C.

12. The method according to claim 11, wherein the high shear rate referred to in step (v) is performed at 3500 rpm or greater.

13. A cosmetic method of obtaining a deodorancy benefit comprising topically applying the deodorant composition according to claim 1 to a surface of the human body.

14. A method of obtaining a skin care benefit comprising topically applying the deodorant composition according to claim 1 to a surface of the human body.

15. A method for reducing malodourous volatile fatty acids and/or thioalcohols comprising topically applying the deodorant composition according to claim 1 to a surface of the human body.

16. The method according to claim 11, wherein step (iv) comprises adding the contents of the second vessel to the stirred contents of the first vessel to form the mixture.

17. The method according to claim 11, wherein step (ii) comprises the substep of filling a third vessel with water; and step (iv) comprises the substep of adding the contents of the third vessel to the mixture at no more than ambient temperature to reduce the temperature of the mixture.

18. The method according to claim 11, wherein step (iv) comprises the substep of cooling the mixture to less than 50° C. before adding fragrance.
